(19) United States
(12) Patent Application Publication
(10) Pub. No.: US 2015/0266986 A1
(43) Pub. Date: Sep. 24, 2015

(54) Multifunctional Hyperbranched Polymers

(71) Applicant: National University of Ireland, Galway, Galway (IE)

(72) Inventors: Wenxin Wang, Galway (IE); Robert Kennedy, Galway (IE); Sean McMahon, Galway (IE)

(73) Assignee: National University of Ireland, Galway, Galway (IE)

(21) Appl. No.: 14/221,106

(22) Filed: Mar. 20, 2014

(51) Int. Cl. C08F 222/20; C08J 3/24; A61L 26/00; C08F 220/68

(52) U.S. Cl. CPC C08F 222/20; C08F 220/68; C08J 3/24; A61L 26/008; A61L 26/0023; A61L 26/0066; C08J 2333/14; C08J 2335/02; C08J 2305/08; A61L 2300/404

(57) ABSTRACT

A hyperbranched copolymer comprising PEG-based monomers is described wherein the hyperbranched copolymer comprises a double bond content in the range 5 mol % to 50 mol %. RAFT (Reverse Addition-Fragmentation chain Transfer) polymerisation is used to synthesise the PEG-based hyperbranched copolymer described herein. The hyperbranched copolymer can be used for the preparation of antibacterial hydrogels.

### FIELD OF THE INVENTION

The present application relates to hyperbranched polymers which may be used for a range of applications, in particular for the preparation of hydrogels. PEG-based (polyethylene glycol) hyperbranched copolymers are described.

### BACKGROUND OF THE INVENTION

Hydrogels and their use in biomedical applications has become an area of much research in recent years. Hydrogel dressings are designed to hold moisture on the surface of a wound, providing an ideal environment for both cleaning of the wound and allowing the body to rid itself of necrotic tissue. These dressings are accepted by a high percentage of patients as they promote a reduction in pain through cooling effects. Additionally, hydrogels are nonreactive with biological tissue, permeable to metabolites and non-irritant.

Wound infection occurs due to the colonization of various anaerobic and aerobic bacteria in an almost perfect proliferating environment of wound tissue. However, this is not the only case. Wound infection due to diseases or conditions such as diabetes, immunosuppression or EB (Epidermolysis bullosa) can influence the bacterial balance in the body. In these cases infection ensues when the bacterial bioburden reaches a level which the host cannot withstand.

The wound healing market represents a 20 billion dollar market. With declining antibiotic efficiency and development of “super bugs”, combined with an aging population, there is now a demand for new advanced treatments. The condition of the wound and the repair of such depend greatly on keeping the wound pathogen free. Traditionally wound care has been developed as a mechanical barrier to adverse pathogens.

Currently there are a number of hydrogel wound care dressings available on the market. These hydrogel wound care dressings can be categorized based on their form of application. Hydrogels can be applied as either an amorphous gel or as elastic solid sheet. They can also be used in combination with gauze dressing and fibres. An example of hydrogel dressings that are currently on the market are Intrasite gel and Flexigel sheet dressing available from Smith and Nephew. These dressings are clinically proven to facilitate gentle, effective autolytic debridement to prepare the wound bed in all types of wounds. As such they are not associated with inflammation, foreign body reactions, tissue necrosis, or extensive fibrosis. One such product available from 3M is called 3M™ Tegaderm™ Hydrogel Wound Filler. This is a sterile, non-preserved, amorphous hydrogel formulated to provide moisture to a dry wound, maintain a moist wound environment which enhances wound healing and helps prevent wound desiccation, and promote autolytic debridement of a dry wound by providing moisture to devitalized tissue. The product comprises water, propylene glycol, guar gum and sodium tetraborate.

US 20120231072 A1 describes thermoresponsive hydrogel compositions. It describes the use of a biocompatible monomer and/or polymer having an amino acid side chain which can form a hydrogel at physiological temperature. The hydrogel can incorporate or encapsulate a treatment agent such as a drug, biomolecule and/or nanoparticle. The amino acid side chain comprises lysine, tyrosine, serine, cysteine, proline, or combinations or derivatives thereof. The polymer forming the hydrogel comprises PNIPAAm (poly(N-isopropylacrylamide)) and PEG-DA (poly(ethylene glycol)diacrylate). The use of poly(ethylene glycol)diacrylate, bisacrylamide, or dithiol functionalized molecules as crosslinkers is described. The treatment in physiochemical state 1(solution) is administered to a mammal at body temperature whereby hydrogel formation occurs achieving physiochemical state 2 with the resulting product having a solid state.

U.S. Pat. No. 8,333,743 B2 relates to antimicrobial bandage material comprising superabsorbent and non-superabsorbent layers. It describes methods and compositions for materials having a non-leaching coating that has antimicrobial properties. The coating is applied to substrates such as gauze-type wound dressings. Covalent, non-leaching, non-hydrolyzable bonds are formed between the substrate and the polymer molecules that form the coating. A high concentration of anti-microbial groups on multi-length polymer chains and relatively long average chain lengths, contribute to an absorbent or superabsorbent surface with a high level antimicrobial effect. The antibacterial bandage materials consist of PEG-DA as a crosslinker. The antibacterial bandage consists of polymers of a first monomeric quaternary ammonium salt, second monomeric quaternary ammonium salt and at least one cross linking agent.

U.S. Pat. No. 8,343,535 B2 relates to a wound healing dressing and methods of manufacturing the same. A hydrogel dressing for covering and treating a wound and a method for the preparation of same is described. The hydrogel dressing includes a matrix structure of a cross-linked mixture consisting of polyethylene glycol and polypropylene glycol and an elastic sheet coated with an elementary metal or ionic metal such as silver (nanocrystalline form), zinc or copper. The mixture comprises a hydrophilic polymer (polyethylene glycol and polypropylene glycol), about 0.5 to about 5 wt % of a photocatalyst agent (comprised of an ultra-fine titanium dioxide) and at least 80 wt % of water based on the total weight of the mixture.

One area where research has been focussed is on the production of hyperbranched copolymers using DE-ATRP (deactivated enhanced atom transfer radical polymerisation). For example a thermoresponsive PEGDA-MEO2-PEGMEMA copolymer formed by DE-ATRP has been reported. It reacts to temperature conditions of 37° C. to crosslink to form a hydrogel. However, this feature can limit the workability of the system for certain applications, for example in a clinical setting.

Despite developments in the field, there remains a need for improved bio-compatible hyperbranched polymers, in particular those that can be used for the preparation of hydrogels for use in biomedical applications.

### SUMMARY OF THE INVENTION

Accordingly, in one aspect, the present teaching provides a hyperbranched copolymer comprising PEG-based monomers wherein the hyperbranched copolymer comprises a double bond content in the range 5 mol % to 50 mol %. The copolymer comprises a high degree of free vinyl functional groups. The hyperbranched copolymer according to the present teaching may comprise a double bond content in the range 10 mol % to 25 mol %, preferably at least 24 mol %.

The copolymer described herein comprises a high degree of hyperbranching within its structure. The hyperbranched copolymer comprises a degree of branching in the range 5 mol % to 30 mol %, preferably at least 27 mol %.

The present teaching provides a branched or hyperbranched polymer having controlled molecular weights. Suitably, the molecular weight and the composition (that is, the number and ratio) of sidearm chains can be controlled or adjusted by appropriate choice of the co-monomer composition used to provide the polymer, by for example, a controlled living polymerisation process.

The hyperbranched copolymer according to the present teaching comprises PEG-based monomers having molecular weights in the range 1000 Da to 500 kDa.

The copolymer described herein comprises end functional groups which are distributed evenly within the architecture of the hyperbranched copolymer without a molecular core. Therefore the functional vinyl groups are easily accessible and are not grouped within the structure. It will be appreciated that the end group functionality may be adjusted to a degree which is appropriate for the end application.

The PEG based monomers may comprise end functional groups selected from the group consisting of acrylate, methacrylate, hydroxyl, carboxyl, amine, thiol, aldehyde, azide and sulphone, for example.

In a preferred aspect of the present teaching, the PEG-based monomers may be selected from the group consisting of PEGDA ((poly)ethylene glycol)diacrylate), PEGAcryate ((poly)ethylene glycol)acrylate), PEGMEMA (poly(ethylene glycol) methyl ether methacrylate), PEGDMA (poly(ethylene glycol)dimethacrylate) and PEG methacrylate (poly(ethylene glycol) methacrylate).

In a particularly preferred aspect, the hyperbranched copolymer comprises PEGDA-PEGMEMA copolymer.

The present teaching provides a hyperbranched copolymer wherein one or more agents can be conjugated to the end functional groups. The agent may be selected from the group consisting of biomolecules, proteoglycans, fibres, glycoproteins, non-proteoglycan polysaccharides, antimicrobial agents and drugs, for example.

The hyperbranched copolymers according to the present teaching are crosslinkable in situ to form a hydrogel. An advantage of the copolymer described herein is that it is capable of cross-linking in situ without the need for extra cross-linking agents or specific environmental conditions. It facilitates rapid gelation of hydrogel networks in situ due to the high degree of free vinyl functional groups within the polymer structure compared to known systems.

A further advantage of the copolymer system described is that it is not sensitive to light. The hyperbranched copolymer according to the present teaching is stable. It has a shelf life of at least 12 months. It has a longer shelf life compared to known copolymer systems synthesised by DE-ATRP for example. It is therefore highly desirable for a wide range of applications.

In a further aspect, the present teaching provides a process for the synthesis of a hyperbranched PEG-based copolymer. The process comprises copolymerising PEG-based monomers using RAFT (Reverse Addition-Fragmentation chain Transfer) polymerisation. In a preferred aspect, the PEG-based monomers comprise PEGDA and PEGMEMA.

The process suitably comprises the use of a RAFT agent selected from Dithiobenzoates, Trithiocarbonates and Dithiocarbamates. For example, 2-cyanoprop-2-yl dithiobenzoate may be used as the RAFT agent in the process described herein.

RAFT polymerisation eliminates the use of metal and ligand compounds. It therefore significantly reduces purification time and the workability of the resulting copolymers is highly desirable for fabrication of hydrogels compared to copolymers prepared by other living controlled polymerisation such as DE-ATRP, for example. The process according to the present teaching allows control over the degree of hyperbranching and number of active vinyl functional groups. The monomer feed ratios of PEGDA and PEGMEMA may be varied in order to obtain a copolymer having the desired functionality.

The molar ratio of cross-linker may be up to 75% of the total feed ratio of monomers. For example, vinyl monomers such as PEGDA or EGDMA (ethylene glycol dimethacrylate) may be used.

In a preferred aspect, the initial monomer feed ratio of PEGDA may be in the range 25 mol % to 75 mol %, preferably 50 mol % of the total feed ratio of monomers. This molar ratio can be used without causing gelation.

The monomer feed ratio of PEGMEMA may be in the range 25 mol % to 75 mol %, preferably 50 mol % of the total feed ratio of monomers.

The present teaching provides a process for the preparation of a hyperbranched copolymer comprising PEGDA-PEGMEMA copolymer. The process may further comprise the step of combining the PEGDA-PEGMEMA copolymer with a cross-linking agent to form a hydrogel.

The crosslinking agent may be selected from the group consisting of thiolated hyaluaronan, pentaerythritol tetrakis 3-mercaptopropionate (QT), chitosan, gelatine, thiolated chitosan, fibrinogen or any natural thiolated biomolecule.

The selection of appropriate monomers and their composition can lead to the formation of a hydrogel in situ at physiological conditions via chemical cross-linking. Thus, the formation of hydrogels by the system described herein is not affected by temperature.

It will be appreciated that enzyme mediated cross-linking may also be used.

In a still further aspect, the present teaching provides a two part hydrogel composition comprising (i) a crosslinking agent and (ii) a polymeric solution comprising a hyperbranched PEG-based copolymer wherein the hyperbranched copolymer comprises a double bond content in the range 5 mol % to 50 mol %.

The polymeric solution preferably comprises an in situ cross-linkable PEGDA-PEGMEMA copolymer according to the present teaching. The crosslinking agent may be selected from the group consisting of thiolated hyaluaronan, pentaerythritol tetrakis 3-mercaptopropionate (QT), thiolated chitosan, fibrinogen, gelatine, chitosan or any natural thiolated biomolecule.

The polymeric solution may further comprise an agent selected from the group consisting of biomolecules, antimicrobial agents, antibacterial agent, anti-fungal agent, anti-viral agent, drug, agent for the regeneration of tissue, fibrinogen or a gene.

The antibacterial agent may be selected from the group consisting of silver nanoparticles, colloidal silver, silver sulfadiazine, antibiotics, honey, copper, zinc, hydrogen peroxide and antimicrobial peptides (AMP's).

The present teaching provides a hydrogel comprising a hyperbranched PEGDA-PEGMEMA copolymer as described herein. Hybrid hydrogel networks can be prepared by combining the cross-linkable copolymer of PEGDA-PEGMEMA with thiol-modified hyaluronic acid, for example Glycosan-Hystem™, HA-SH.

The polymer according to the present teaching is versatile and can be used in a variety of applications. The PEG-based system described herein is highly soluble and biocompatible. For example, it can be used in the preparation of an injectable, in situ cross-linking, hybrid hydrogel system by using “click” thiol-ene reaction.

The hydrogel itself can be customised and modified to a great extent achieving a wide range of properties and functions. The nature of the hydrogel can be controlled and manipulated to create a desirable product. For example, the hydrogel can be customised to make it transparent. This transparency could be achieved by a process of purification and removal of the RAFT agent post polymerisation step. The polymer could be purified to remove all coloured agents by methods of dialysis and precipitation and the remaining polymer could be made transparent. This polymer in turn would produce a transparent hydrogel. This allows the state of a wound to be assessed without removing the dressing. Furthermore the hydrogel can be combined with external agents such as an antibacterial agent, for example, to create a combined effect.

Suitably, the hydrogel may further comprise an antibacterial agent. The antimicrobial hydrogel may be used for wound care and may be applied to a range of wounds. It may work effectively with the body's innate healing mechanisms. The hydrogel may be used in wound dressings or applied directly to an infected area as a gel.

In a yet further aspect, the present teaching provides a wound dressing comprising a hydrogel as described herein.

The present teaching further provides the use of a two part hydrogel composition as described herein or a hydrogel as described herein for topical treatment of a wound or infection. The two part hydrogel composition or hydrogel may be used as an antibacterial composition.

The two-part hydrogel composition according to the present teaching or the hydrogel described herein may be used for the treatment of mastitis.

The present teaching provides a method of treating a wound or infection comprising the steps of (i) applying a polymeric solution comprising an in situ cross-linkable PEGDA-PEGMEMA copolymer as described herein to said wound or infection and (ii) adding a cross-linking agent to said solution such that a hydrogel is formed in situ. The polymeric solution may further comprise an anti-bacterial agent such as silver, for example.

In an alternative method, the components of the two-part hydrogel composition described herein may be combined to form a hydrogel before application to a wound or infection. In this method, the cross-linking agent may be added to the polymeric solution comprising the in-situ cross-linkable PEGDA-PEGMEMA copolymer such that a hydrogel is formed. The hydrogel thus formed can then be applied to the wound or site of infection.

In a further aspect, the hyperbranched copolymer system according to the present teaching, or synthesised by the process described herein, may be used as a drug or gene delivery vehicle or as a diagnostic tool.

The hyperbranched copolymer according to the present teaching or synthesised by the process described herein may also be used as a cross-linking agent. For example, it may be used as a cross-linking agent in the preparation of a 3 dimensional printable material.

The highly branched copolymer may be used in 3D printing as a rapid cross-linker due to its highly branched structure. For example, one of the main limitations within 3D printing technology is soft materials. There is a need for 3 dimensional printable soft materials. These are especially valuable with respect to anatomical and biological efforts, for example in the field of generation of and replacement of organs, scaffolds or soft tissue reinforcements. Currently available systems present limitations for example, speed limitation of gelation or cross-linking. Current soft materials used in 3D printing are proving difficult to work with due to the time taken for layers of the material to cross-link. This problem presents feasibility issues with printing soft tissue constructs as the material cannot support layers prior to curing. The hyperbranched technology described herein has significant advantages in this field with a faster curing speed thus allowing for more efficient 3D printing. Current technology for soft printable materials faces significant challenges with curing speed as subsequent layers need to wait until the previous layer has cured so as to support the application of a new layer. Linear polymers do not hold the same potential for speed of curing that a hyperbranched polymer according to the present teaching has. Therefore this polymer may be used as a 3D printable material to target these drawbacks.

The hyperbranched polymer system described herein may be used in combination with other cross-linking systems (for example, injection printing with UV light curing) to develop an improved 3D printing system. These systems may be used for the preparation of soft tissue biological materials such as, for example, skin or vascular system components.

The hyperbranched copolymer according to the present teaching may also be used as an additive. It may be used in the field of additive technology where for example, additives are used to alter the inherent properties of a material. For example, different blends of the copolymer could be added to a material in order to alter the viscosity of the material. The copolymer could also be added to a composite material in order to give different mechanical or chemical properties to the material.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic of the process for the synthesis of a multifunctional hyperbranched copolymer according to the present teaching;

FIG. 2 is a schematic illustration of Ag hydrogel cross linking and in situ hydrogel formation;

FIG. 3 shows GPC traces from RI detector for copolymer PEGDA-PEGMEMA, final molecular weight of 296,000 g/mol, a polydispersity of 7.5 after the reaction time of 7.5 hrs;

FIG. 4 is 1H-NMR analysis of PEGDA-PEGMEMA copolymer in CDCl3 (7.2 ppm);

FIG. 5 is a graph showing the release of Silver Sulfadiazine from the hydrogel matrix over time;

FIG. 6 is a graph showing the concentration of Silver Sulfadiazine released vs. \sqrt{Time} Hr⁻¹;

FIG. 7 is a graph showing the results of an absorbance study of silver nanoparticle hydrogels—Escherichia coli. A=0.01%, B=0.005%%, C=0.0025%, D=0% and E=Bacteria without hydrogel;

FIG. 8 is a graph showing the results of an absorbance study of silver nanoparticle hydrogels—Pseudomonas aeruginosa. A=0.01%, B=0.005%%, C=0.0025%, D=0% and E=Bacteria without hydrogel;

FIG. 9 is a graph showing the results of an absorbance study of silver nanoparticle hydrogel—Staphylococcus aureus A=0.01%, B=0.005%%, C=0.0025%, D=0% and E=Bacteria without hydrogel;

FIG. 10 is a graph showing disk diffusion zones, i.e. zones of inhibition for the various species tested after 72 hours;

FIG. 11 is a graph showing the results of the absorbance test for Escherichia coli. A=5% SSD, B=1.0% SSD, C=0.1% SSD, D=0% SSD and E=Bacteria alone;

FIG. 12 is a graph showing the results of the absorbance test for Pseudomonas aeruginosa. A=5% SSD, B=1.0% SSD, C=0.1% SSD, D=0% SSD and E=Bacteria alone;

FIG. 13 is a graph showing the results of the absorbance test for Staphylococcus aureus. A=5% SSD, B=1.0% SSD, C=0.1% SSD, D=0% SSD and E=Bacteria alone;

FIG. 14 is a graph showing the results of cell viability studies; Cell Viability day 1. 0.005, 0.01% silver nanoparticle hydrogels. 1.0% silver sulfadiazine hydrogel and 0.0% control hydrogel;

FIG. 15 is a graph showing the results of cell viability studies; Cell Viability day 3. 0.005, 0.01% silver nanoparticle hydrogels, 1.0% silver sulfadiazine hydrogel and 0.0% control hydrogel; and

FIG. 16 is a graph showing the results of cell viability studies; Cell Viability Day 7. 0.005, 0.01% silver nanoparticle hydrogels, 1.0% silver sulfadiazine hydrogel and 0.0% control hydrogel.

### DESCRIPTION OF THE PREFERRED EMBODIMENTS

The present teaching provides biocompatible and cross-linkable hyperbranched polymers. The polymers described are particularly suitable for use in the preparation of hydrogels, such as hydrogel dressings for wound repair, for example.

A hydrogel is a network of polymer chains that are hydrophilic. Hydrogels are highly absorbent natural or synthetic polymers. They possess a degree of flexibility very similar to natural tissue due to their significant water content and have therefore been the focus of much research for biomedical applications.

Wound healing typically occurs through the processes of haemostasis, inflammation, tissue repair and remodelling. However, complications such as diabetic ulcers and arterial ulcers can develop into chronic wounds which are difficult to manage and heal. Therefore, wound infection can be life threatening and can be traumatic and debilitating. Recognition of infection is relatively easy with signs of redness, pain, heat, erythema and oedema in acute wounds while chronic wounds have a pungent smell, the patient experiences severe pain and wound tissue death/necrosis.

The use of antibacterial/antimicrobial hydrogel dressings such as described herein, offers many advantages over the traditional wound dressings currently employed. The wound is provided with a moist environment which promotes re-epithelialisation and cell growth. The hydrogel acts as a mechanical barrier as it completely covers the wound from the external environment preventing invading fungi, viruses, bacteria and pathogens from entering the wound site. The incorporation of an active antibacterial agent debilitates any attempt to disrupt the natural healing process. Additionally, by covering the wound with a moist dressing contraction of the wound is prevented leading to less scar formation, increasing angiogenesis and thus increased healing rates in patients.

In one aspect, the present teaching provides a platform for a polymer based approach for wound treatment that addresses the challenges presented by known systems for wound treatment. The present teaching uses RAFT (Reverse Addition-Fragmentation chain Transfer) polymerisation to control the rate of polymer synthesis to achieve a polymer having a particular molecular weight and desirable number of end functional groups. The copolymer according to the present teaching allows for enhanced workability, a high degree of functionality due to the high number of vinyl functional groups and a long shelf life.

The PEG based copolymer according to the present teaching comprises a hyperbranched polymer. The PEG based copolymer system described herein is biocompatible and highly soluble.

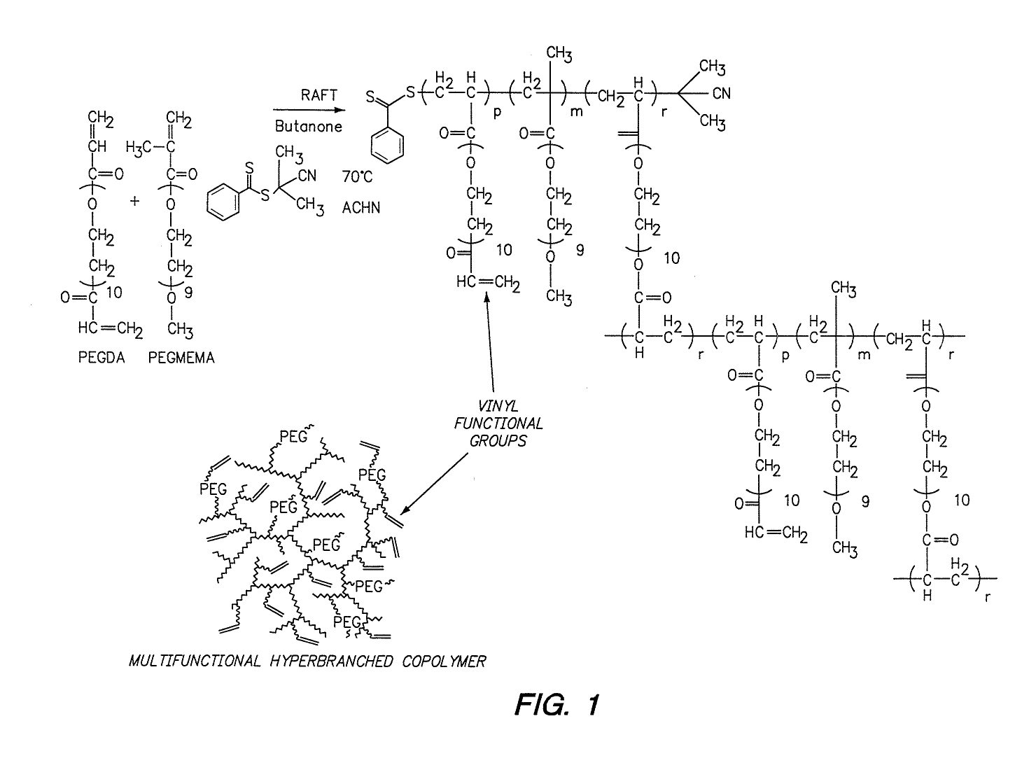

With reference to FIG. 1, the multifunctional hyperbranched copolymer according to the present teaching, synthesised via RAFT (Reverse Addition-Fragmentation chain Transfer) polymerisation using PEGDA-PEGMEMA, can be readily combined with an antibacterial agent in solution and cross-linked with a variety of cross-linkers to form a hydrogel in situ (under physiological conditions). The hydrogel according to the present teaching demonstrates antibacterial activity.

RAFT polymerisation can be used to synthesise polymers with controlled molecular weight. RAFT polymerisation was used in the process according to the present teaching because it is tolerant of a very wide range of functionality in the monomer and solvent. RAFT polymerisation can also be carried out over a wide temperature range.

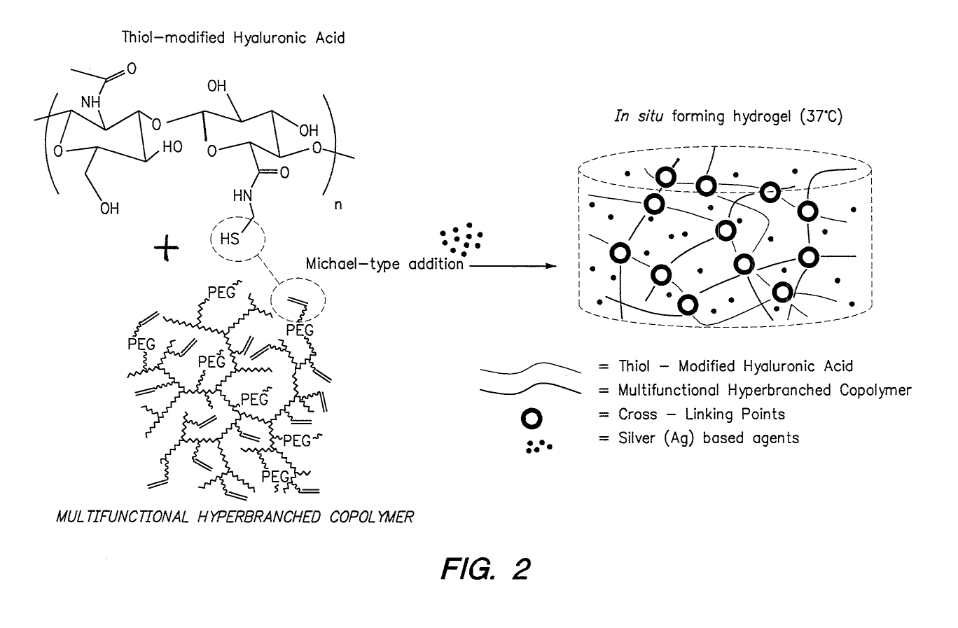

With reference to FIG. 2 a schematic illustration of Ag hydrogel cross linking and in situ hydrogel formation is shown. This was carried out through thiol Michael type addition using thiol modified hyaluronic acid. This is a reaction between the thiol and vinyl groups to cross link in physiological conditions. However multiple crosslinkers can be incorporated to form hydrogel in the place of hyaluronic acid. Enzyme mediated crosslinking can be carried using biomolecules including gelatine, chitosan and fibrinogen.

The invention will be described in more detail below with reference to the Examples.

### Examples

### Synthesis of Copolymer PEGDA-PEGMEMA

Using the RAFT polymerisation approach a multifunctional hyper branched copolymer comprising PEGDA-PEGMEMA monomers was obtained. Through the use of Gel Permeation Chromatography (GPC) analysis during polymerisation the monomer conversion, molecular weight and polydispersity of the copolymer was inspected until desirable results were yielded. The final characterisation of the copolymer was investigated by H-NMR analysis.

### Materials

The monomers poly(ethylene glycol) methyl ether methacrylate (PEGMEMA, Mn=500 g mol-1) and poly(ethylene glycol)diacrylate (PEGDA, Mn=575 g mol-1) were purchased from Sigma-Aldrich. 1′1-azobis(cyclohexane-carbonitrile) (Sigma-Aldrich, 98%) was used as an initiator. The RAFT agent, 2-cyanoprop-2-yl dithiobenzoate was purchased from Sigma Aldrich (See below). Butanone (HPLC grade, Aldrich), hexane (HPLC grade, Aldrich), tetrahydrofuran (THF, Aldrich) chloroform-d (CDCl3 Aldrich), diethyl ether (HPLC grade, Aldrich). All reagents and solvents were used without further purification.

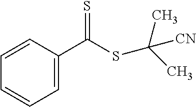

### 2-cyanoprop-2-yl dithiobenzoate

### Polymerisation

PEGMEMA-PEGDA copolymers were synthesized by the copolymerization of PEGMEMA (Mn=500 g mol-1) and multivinyl branching monomer PEGDA (Mn=575 g mol-1) via Reverse Addition-Fragmentation chain Transfer (RAFT) polymerisation. Briefly, the copolymers were prepared in butanone at the concentration of monomer to solvent volume ratio being 1:3 in a 250 ml round bottomed flask. The initiator (1′1-azobis(cyclohexane-carbonitrile) was added (100:0.8, monomer:initiator mol ratio) and followed by the RAFT agent at a mol ratio of 1.6:100 with respect to the monomers employed. The solution was bubbled with argon for 20-25 minutes to remove any oxygen. The reaction was conducted at 70° C. in an oil bath while being stirred at 700 rpm until the desired polymer molecular weight, conversion and polydispersity was acquired (monitored by Gel Permeation Chromatography—GPC). To terminate the polymerisation, the stopper was removed and exposing the reaction to oxygen and cooling the flask rapidly in water.

### Purification

### Precipitation

The polymer was diluted with butanone (Vol % 1:1) and dropped into a mixture of hexane and diethylether with volume ratio of 1.3:1, the total volume of which was 2000 mL. The mixture was stirred rapidly throughout the process. Once the polymer mixture was completely dropped into the mixture of hexane and diethyl ether, the spinner was switched off and allowed to settle for 2 hours. After settling, the solution was poured off. The accumulation of polymer on the bottom of the glassware resulted. This was then covered with aluminium foil in a fume hood for approx. 10 minutes to allow butanone to evaporate from the precipitated mixture. The polymer formed at the bottom of the glassware accumulated in what can be visually described as a sticky gel like paste.

This resulting product was then re-dissolved with 100 mL of deionised water. It is preferable to use the least amount of water possible as the less water used, the less time will be needed to freeze dry the copolymer. This precipitation process eradicates excess PEGDA and PEGMEMA from the final yield of the copolymer.

### Dialysis

Once the polymer was re-dissolved in water it was then dialyzed in deionized water by transferring it into a spectrum dialysis membrane, molecular weight cut off 6000-8000 μm pore size. This process was carried out for 2 days at 4° C. in order to remove remaining excess monomers.

### Freeze Drying

The polymer obtained after dialysis was transferred to wide based beakers for easy access to the resulting product and to increase the exposed surface area for more efficient freeze drying. The polymer was freeze dried at −80° C. and stored at −20° C. until further analysis.

### Characterization of PEGMEMA-PEGDA Copolymers

Characterization of the copolymers was carried out by 1H-NMR and Gel Permeation Chromatography (GPC). Weight average molecular weight (Mw), number average molecular weight (Mn) and polydispersity (PDI, Mw/Mn) were obtained by GPC (Aligent, PL-GPC50) with RI detector. The GPC eluted THF from columns (2× Agilent PLgel, 5 μm, Mixed C 300 mm×7.5 mm) and calibration was carried out using poly(methyl methacrylate) standards. Analysis and calibration was carried out at 40° C. at a flow rate of 1 mL/min. 1H NMR was carried out on a 300 MHz Bruker NMR with Delta NMR processing software. The chemical shifts were referenced to the lock chloroform (CDCl₃).

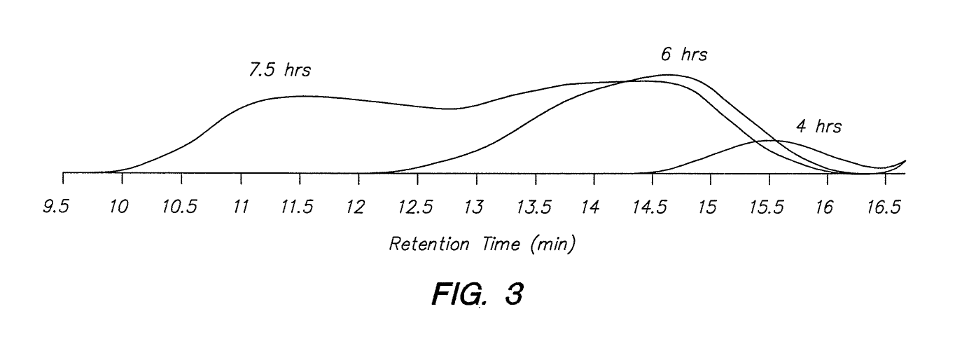

With reference to FIG. 3 controlled/living growth of the copolymer chains was demonstrated with a characteristic shift in the GPC trace from the right to left throughout the reaction at 4 hrs, 6 hrs and 7.5 hrs, while molecular weights increased from 6 kDa to 36 kDa and to 296 kDa respectively. The formation of a hyperbranched structure within the copolymer was displayed where the polydispersity (PDI) at the early stages of polymerisation was narrow (1.2) and broadening significantly to 7.5 at the later phase of the reaction.

TABLE 1
Copolymerisation of Polyethylene glycol diacrylate
(PEGDA - 575 g/mol) and polyethylene methyl ether
methacrylate (PEGMEMA - 500 g/mol) via RAFT polymerisation

| | I:RAFT | SR | | Monomer | Mw | |
|---|---|---|---|---|---|---|
| Entry | Agent:M1:M2^{a} | (v/v)^{b} | RT^{c} | conversion (%)^{d} | (kDa)^{e} | PDI^{f} |
| 1.1 | 0.8:1.6:50:50 | 01:03 | 4 | 14 | 6.5 | 1.25 |
| 1.2 | 0.8:1.6:50:50 | 01:03 | 6 | 42 | 36 | 1.91 |
| 1.3 | 0.8:1.6:50:50 | 1.3 | 7.5 | 57 | 296 | 7.24 |
| 2 | 0.8:1.6:75:50 | 01:03 | 10 | 52 | 370 | 7.12 |
| 3 | 0.8:1.6:25:50 | 01:03 | 7.5 | 57 | 300 | 7.5 |

^{a}I - Initiator (ACHN):RAFT Agent - (2-cyanoprop-2-yl dithiobenzoate), M1:M2 -Monomer feed ratio (PEGDA (M1):PEGMEMA (M2)),
^{c}Reaction time,
^{d}Monomer conversion estimated from copolymer and monomer peaks in GPC traces,
^{e}M_{w} - Weight Average molecular weight (1 kDa = 1,000 g/mol),
^{f}Polydispersity index (M_{w}/Mₙ)

### NMR Analysis:

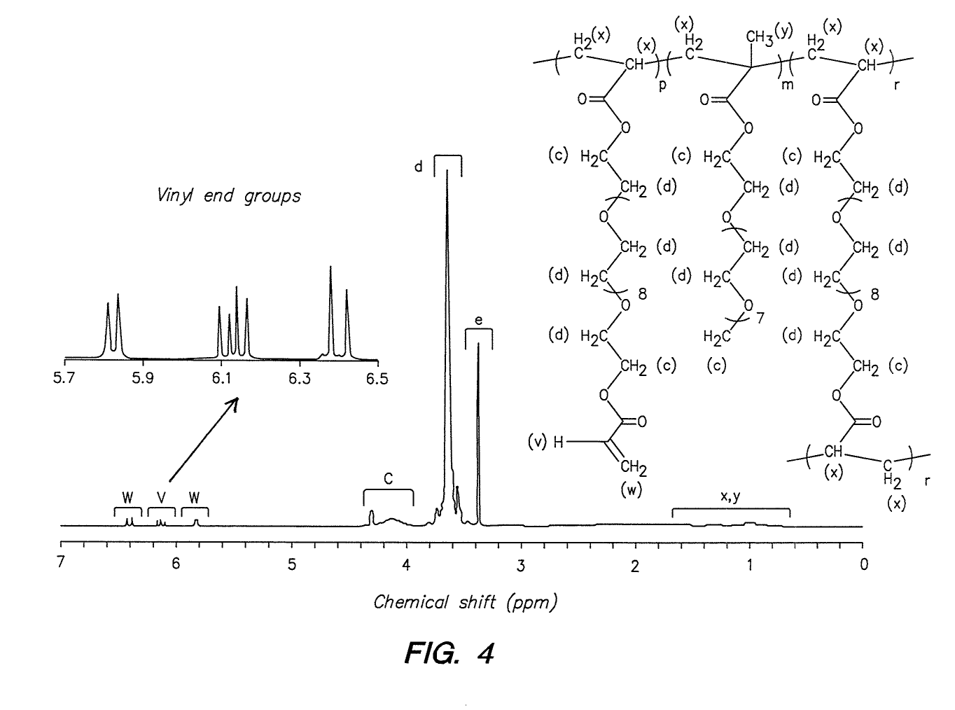

Further characterization of the hyperbranched structure and multiple functional end groups of the copolymers was conducted using ¹H-NMR analysis (FIG. 4). The functionality of the copolymers was demonstrated in the form of vinyl groups at characteristic peaks between 5.8 ppm and 6.4 ppm (FIG. 4). The copolymer composition was determined by integrating v, c, d and e peaks (FIG. 4). Equations 1-4 outline the calculations:

$$\begin{matrix}p = v & ( 1 ) \\ 2 p + m + 2 r = \frac{( c )}{2} & ( 2 ) \\ 18 p + 15 m + 18 r = \frac{( d )}{2} & ( 3 ) \\ m = \frac{( e )}{3} & ( 4 )\end{matrix}$$

The double bond content and the branching degree of the copolymer were calculated from the following equations (5-6):

$$\begin{matrix}Double bond ( mol \% ) = \frac{p}{( p + m + r )} * 100 & ( 5 ) \\ Hyperbranching Content ( mol \% ) = \frac{r}{( p + m + r )} * 100 & ( 6 )\end{matrix}$$

The polymer composition is outlined in Table 2 showing the double bond content and branching degree of the copolymers. The final degree of vinyl functional groups in the copolymer was open to modification by varying the content of PEGDA during polymerisation. Using a 50:50 feed ratio of PEGDA to PEGMEMA, a copolymer was successfully produced with 22 mol % of vinyl groups and 24 mol % degree of branching. However, decreasing the content of PEGDA monomer within the feed ratio to 25 molar % achieved a reduction in the vinyl functionality to 10% mol while slightly decreasing the branching degree to 20% mol of final copolymer (Entry 3, Table 2). This phenomenon was reiterated by altering the content of PEGDA to 75% molar ratio (Entry 2, Table 2) where the hyperbranching and functionality were increased. The high content of PEG side chains results in the copolymer being highly hydrophilic.

TABLE 2
Copolymer PEGDA-PEGMEMA composition and properties

| | Feed | Polymer | Double bond | Branching degree |
|---|---|---|---|---|
| Entry | Ratio ^{f} | composition ^{g} | content (mol %) ^{h} | (mol %) ⁱ |
| 1 | 50:50:00 | 47:53:0 | 22.1 | 24.5 |
| 2 | 75:50:00 | 51:39:0 | 24.2 | 26.8 |
| 3 | 25:50:00 | 32.8:66.8 | 12.8 | 20 |

^{f} Feed Ratio of Monomers (PEGDA:PEGMEMA),
^{g} Polymer composition determined from ¹H-NMR analysis,
^{h} Double bond content (vinyl end groups) calculated from Eqⁿs (1)-(6),
ⁱ Branching degree within polymer structure calculated Eqⁿs (1)-(6)

### Fabrication and Characterisation of Antibacterial Hydrogel:

FIG. 2 shows a scheme outlining how antibacterial hydrogel networks were developed. Briefly, a copolymer (PEGDA-PEGMEMA) solution was mixed with a solution of thiol modified hyaluronic acid. Hydrogel networks form in situ by a chemical reaction which is known as thiol Michael type addition which is a type of “click” chemistry. The example set out below uses silver as an antibacterial agent.

In order to encapsulate the silver antibacterial agent in the hydrogel a desired concentration was mixed with the polymer solution and crosslinked by adding the hyaluronic acid. This is a reaction between the thiol and vinyl groups to cross link at physiological conditions. However multiple crosslinkers can be incorporated to form hydrogel in the place of hyaluronic acid. Enzyme mediated crosslinking can be carried using biomolecules including gelatine, chitosan and fibrinogen.

Silver sulfadiazine and silver nanoparticles were selected for use as antibacterial agents in the examples herein. It will be appreciated that other antimicrobial agents could also be used.

### Silver Sulfadiazine Release Assay

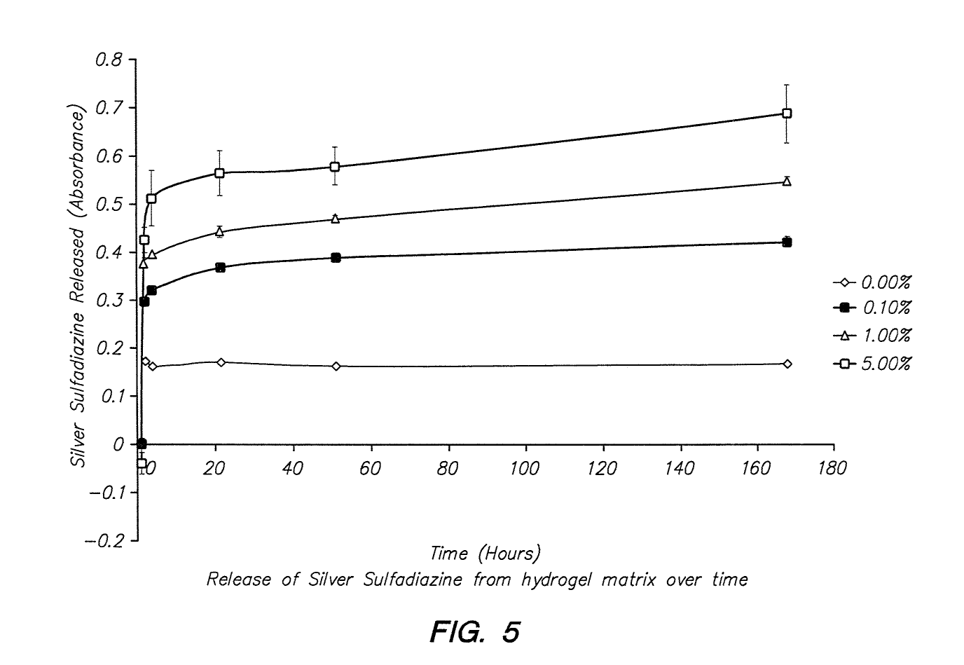

Diffusion studies were conducted to investigate the time release of these particles. This experiment gave a measure of expected release of silver sulfadiazine particles from the hydrogel samples. FIG. 5 shows the release of silver sulfadiazine particles from the gel over an extended timeline. The data for these results were produced by means of an absorbance test.

With reference to FIG. 5, the release assay demonstrates the relationship between concentration of silver sulfadiazine and the amount released over time. After 7 days of continuous rocking motion at room temperature the optical density of PBS solution has risen:
Optical density does not change from 0.165 for 0.0% SSD encapsulated in hydrogel.
From 0.165 to 0.385 for 0.1% SSD encapsulated in hydrogel.
From 0.165 to 0.512 for 1% SSD encapsulated in hydrogel.
From 0.165 to 0.695 for a 5% SSD encapsulated in hydrogel.

Furthermore this study shows that the hydrogel system according to the present teaching steadily releases silver beyond a time point of seven days. It demonstrates release rates comparable to known silver based dressings, such as, for example, (ACTICOAT™ Flex 3 (Smith & Nephew, Milan, Italy), Mepilex® Ag, MölnIycke Health Care, Gallarate, Italy and ACTISORB® Silver 220 (Johnson & Johnson, Rome, Italy) which guarantee sustained antimicrobial action for 3.7 and 7 days. Thus the hydrogel system described herein can compete with advanced currently available silver based dressings.

To relate this absorbance to the actual amount of silver sulfadiazine being released a further investigation was conducted. Concentrations of Silver sulfadiazine were prepared. These concentrations were all below 1%. After preparation of these concentrations their optical density was measured at the same wavelength as this release assay was conducted. A graph was plotted of absorbance vs. % SSD.

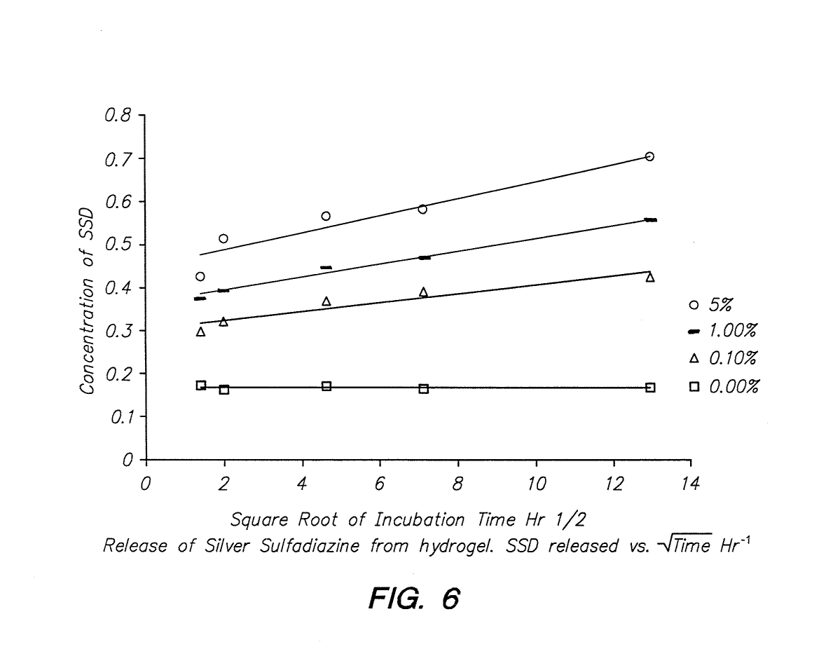

As shown, the release of SSD from hydrogels was very quick at the beginning and then became slower and slower. This SSD release did show a dependency to some extend on the SSD content within the hydrogels. With reference to FIG. 6, when SSD released is plotted against the square root of incubation time, a linear relationship is obtained (except for the initial stages of soaking). This indicates that the release of SSD is controlled by interdiffusion of the particles within the hydrogel itself.

### Anti-Bacterial Studies

The use of silver nanoparticles and silver sulfadiazine as antibacterial agents in the hydrogel according to the present teaching was investigated.

### Disk Diffusion Study—Silver Nanoparticles

Inhibition was noted in all samples containing silver nanoparticles. The control sample displayed no inhibition therefore demonstrating the anti-bacterial activity of silver particles. This inhibition was minor and in bacterial strains Staphylococcus aureus and Escherichia coli this inhibition was hardly recognisable. Furthermore, cell inhibition proved to be proportional to silver nanoparticle density. The sample containing highest density of silver particles inhibited most bacteria growth. The hydrogel proved to keep the nanoparticles within its matrix as there were no signs of diffusion. Results of inhibition are formatted and analysed below.

TABLE 3
Diameter in μm of zones of inhibition.

| | Sample A | Sample B | Sample C | Sample D |
|---|---|---|---|---|
| Sample | 0.01% | 0.005% | 0.0025% | 0.0% |
| 1^{st} sample | 5000 μm | 3500 μm | 2100 μm | 0 μm |
| 2^{nd} | 4000 μm | 3000 μm | 1800 μm | 0 μm |
| 3^{rd} | 5200 μm | 3200 μm | 1000 μm | 0 μm |
| Average | 4730 μm | 3230 μm | 1630 μm | 0 μm |

The results in Table 3 show that as the concentration of silver decreases, the zone of inhibition diameter also decreases. Samples were inspected again at 48 hours. No change was observed aside from increased growth of bacteria.

Inhibition of growth was noted in samples. However, it was recognised that the silver nanoparticles did not diffuse from hydrogel matrix. It was as a direct result of this that diffusion of particles was investigated. It was later concluded that these silver nanoparticles were trapped within the hydrogel matrix and therefore could not diffuse readily.

The results from this study were positive. They showed that the silver nanoparticles encapsulated within the hydrogel did induce a zone of inhibition but only to the area in direct contact with hydrogel samples. The zone of inhibition was proportional to the density of silver nanoparticles present.

### Anti-Bacterial Absorbance Study

Sample readings were taken at 0, 1, 2, 3, 4, 6 and 7 hours. Growth Curves for bacteria were plotted when absorbance measurements were taken from plates including hydrogels. The presence of hydrogel distorts absorbance readings and therefore the results cannot be considered as highly accurate. However, the results are still of interest as they give an indication of the effectiveness of hydrogel inhibition properties. Prior to spectroscopy readings being taken, samples of culture were taken and plated in new 96 well-plates to remove the presence of hydrogel. Two controls were used, firstly a hydrogel sample containing 0.0% silver (D) and secondly a control containing only bacteria culture (E) and no hydrogel.

Analysis of Results from Absorbance Test

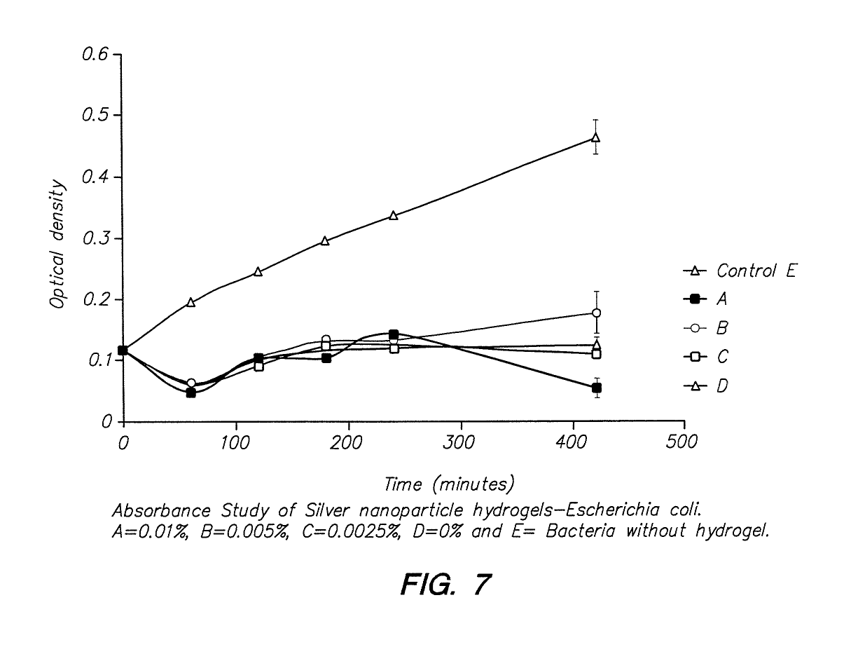

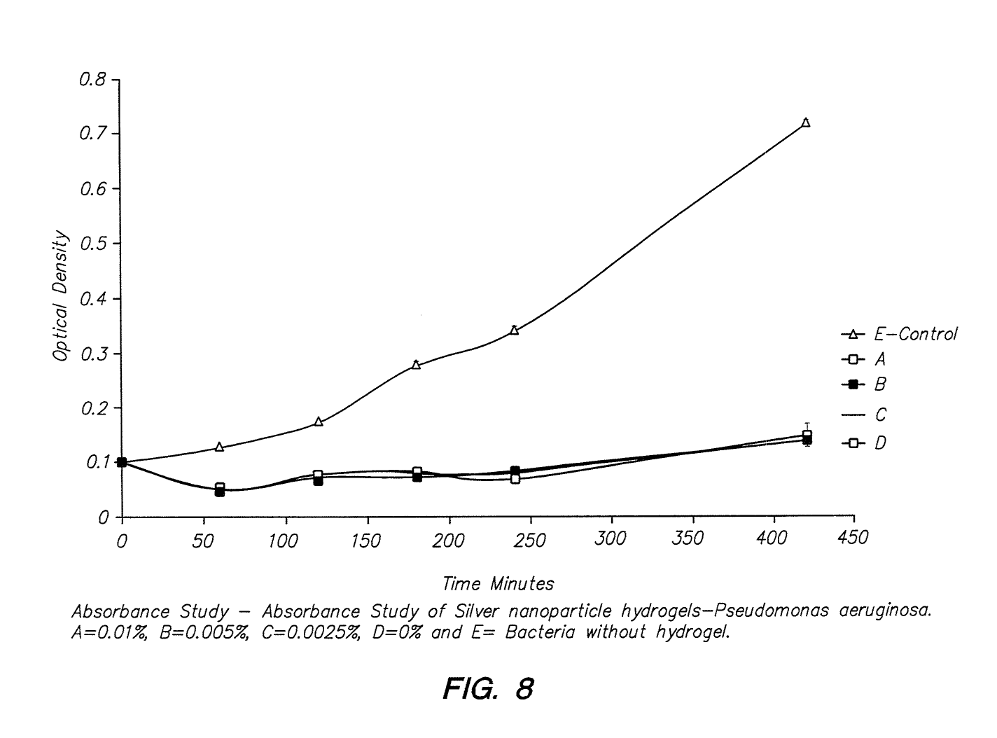

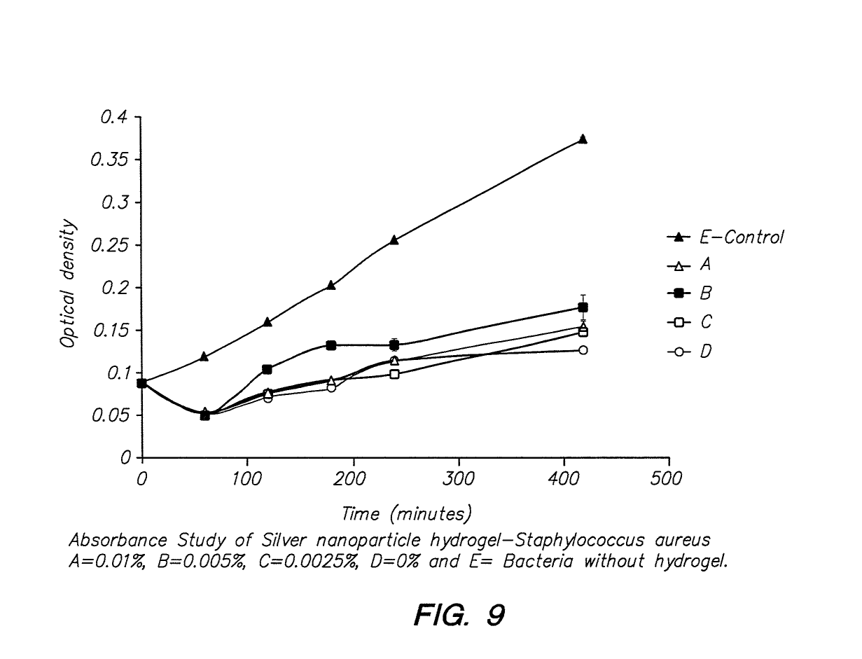

With reference to FIGS. 7-9, the results showed a highly significant reduction in the growth of bacteria which can be linked directly to the presence of hydrogel. The growth of control bacteria (no hydrogel) proved faster than that of the bacteria in contact with the hydrogel. This study has shown synonymously that these silver nanoparticles encapsulated within hydrogel inhibit the growth of a range of gram positive and gram negative bacteria.

Furthermore the results demonstrated a trend of initial dipping and subsequent rising of bacteria growth when exposed to hydrogel samples. This drop can be explained by the initial addition of hydrogel samples. One conclusion is that the surface of these hydrogel samples interacts with bacteria upon contact to inhibit growth further. This is not a lasting effect; it is expected that once the anti-bacterial agent on the surface of hydrogel samples (namely silver nanoparticles) has been used up entirely then these bacterial strains begin to grow once again. This is highly interesting and suggests that the concentration of these silver nanoparticles being used may be too low. Following subsequent research and evaluation, an optimum concentration in the range of 1-2% silver nanoparticles was determined. This range exhibited acceptable toxicity levels while also providing a sustained anti-microbial effect for an acceptable length of time comparable (in some cases longer) to current market products. This optimum concentration is based on experimental results from cytotoxicity evaluation, antimicrobial studies and a detailed market study on the current silver based wound care dressings—typically an optimum concentration of 1% is used however this value could be increased to 2% for particularly resilient heavy duty dressing, for example an infected wound. It also suggests that there is an inherent anti-bacterial property within the hyaluronan present in these gels.

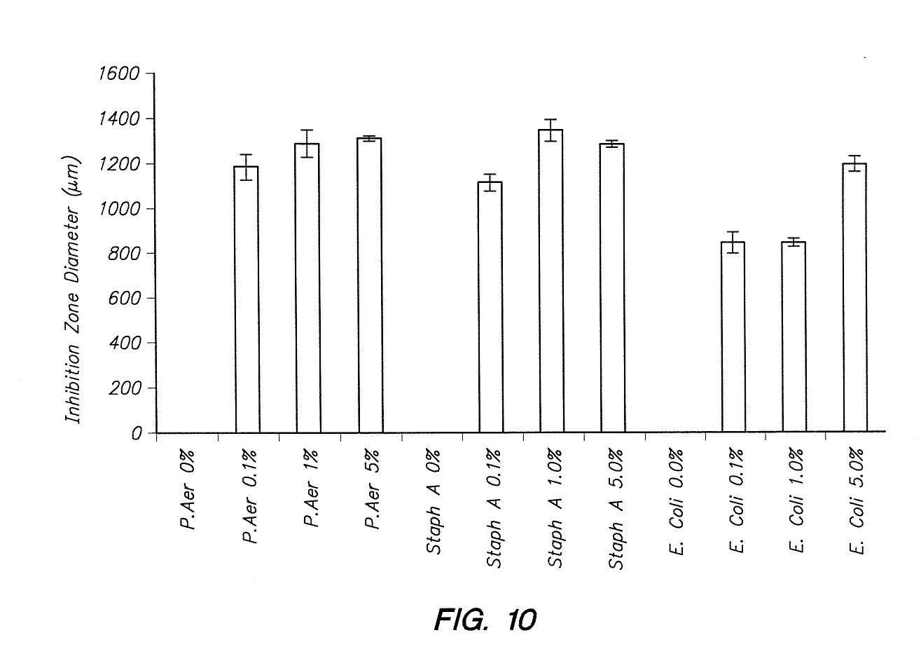

FIG. 10 shows zones of inhibition of samples after 72 hours. All samples containing silver sulfadiazine exhibited significant inhibition.

### Absorbance Test

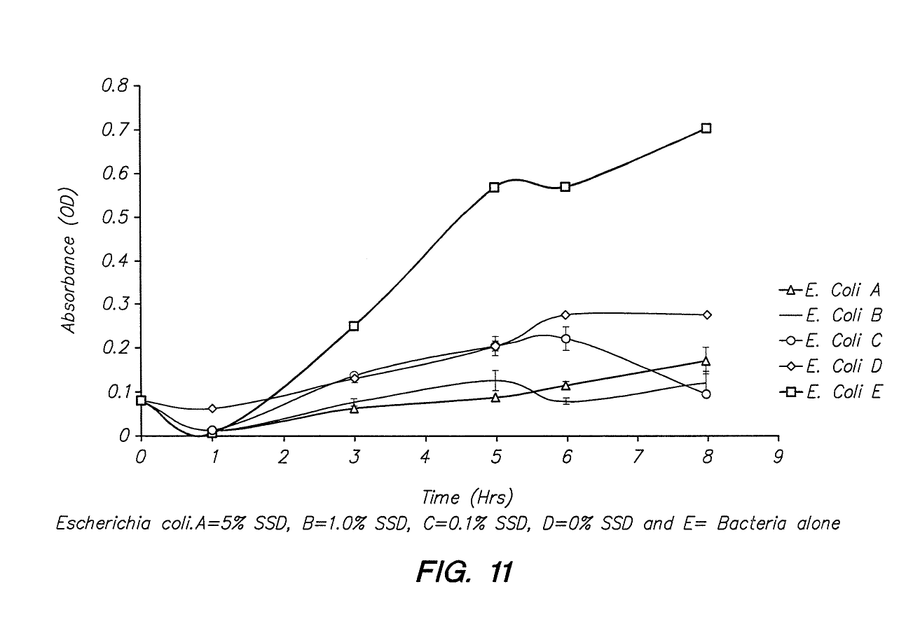

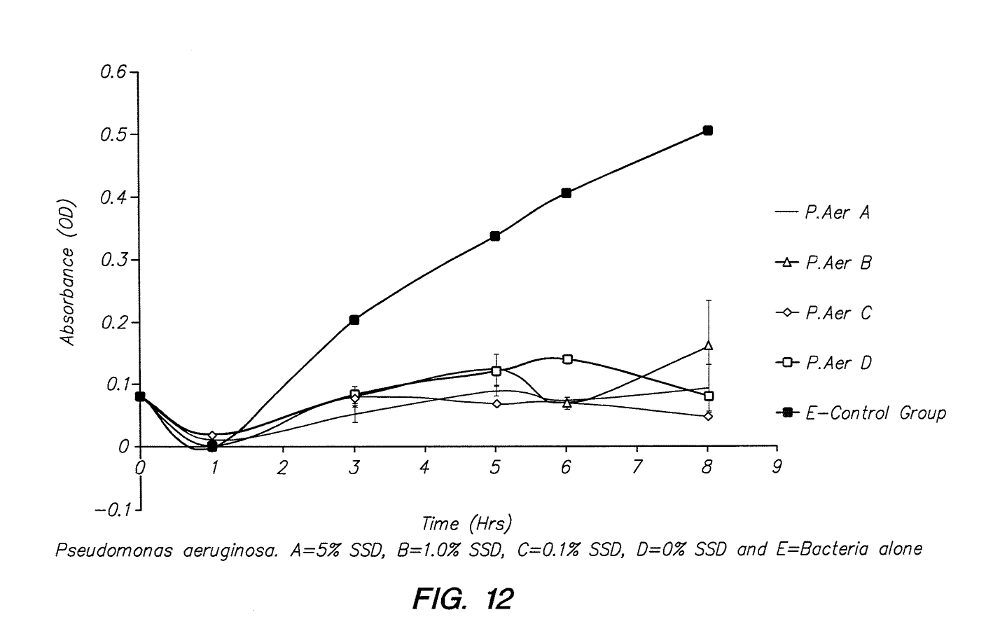

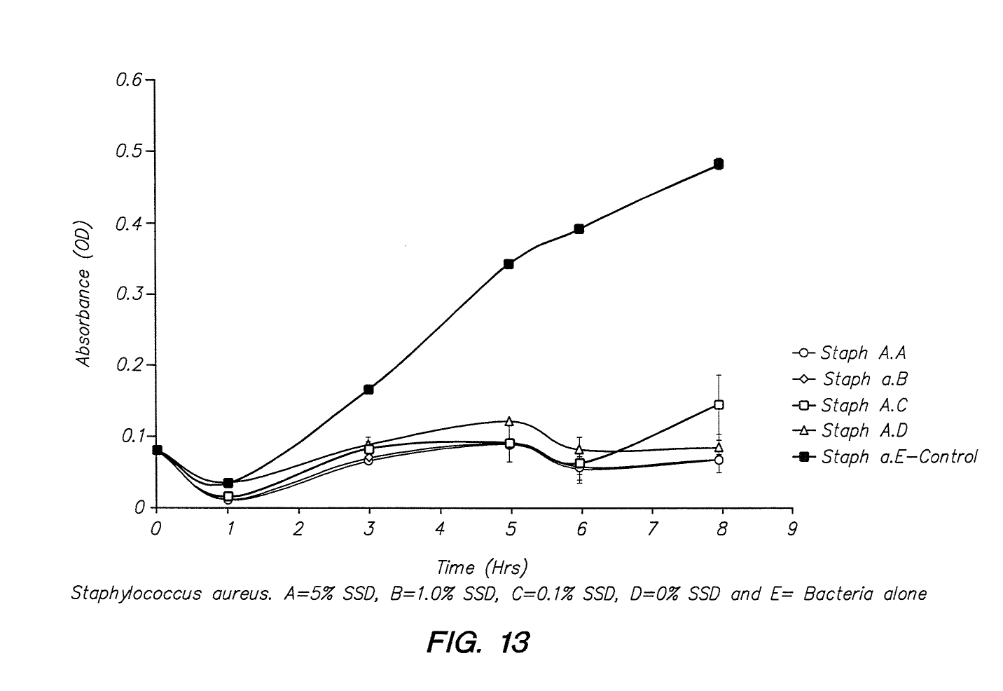

With reference to FIGS. 11 to 13, the absorbance test demonstrates the anti-bacterial efficiency of these silver sulfadiazine particles well. The results show that bacteria growth in the presence of these hydrogels was greatly inhibited. Furthermore the presence of silver sulfadiazine proved to reduce this pathogenic growth further. Since both controls used in this experiment exhibited less inhibition than these SSD samples it can be concluded that these hydrogels allow for the dispersion of silver sulfadiazine. Hydrogel samples showed significant inhibition against all strains of bacteria tested. This inhibition was furthered by the incorporation of these silver sulfadiazine particles.

### Biological Characterisation

### Cytotoxicity

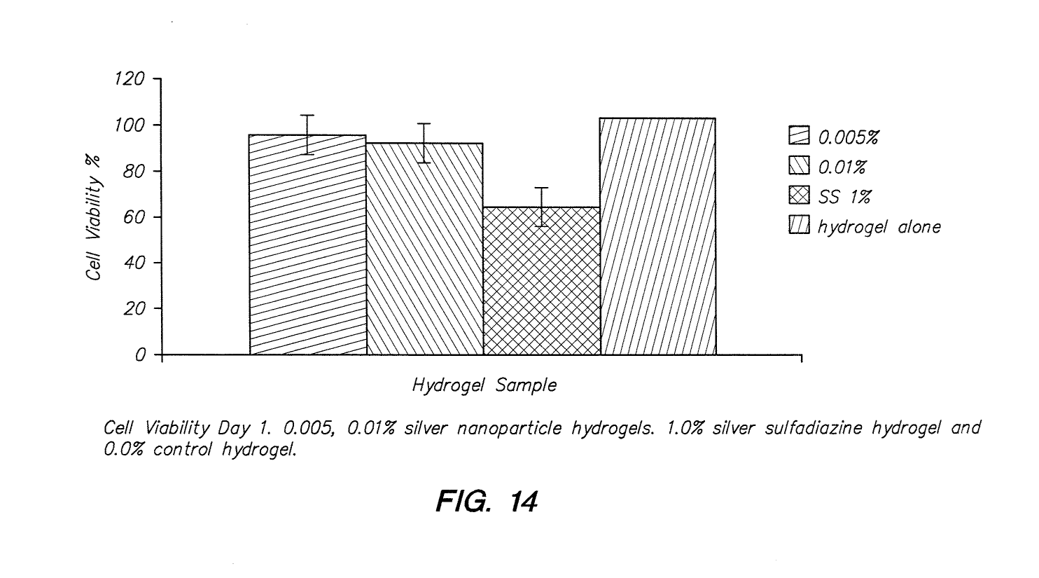

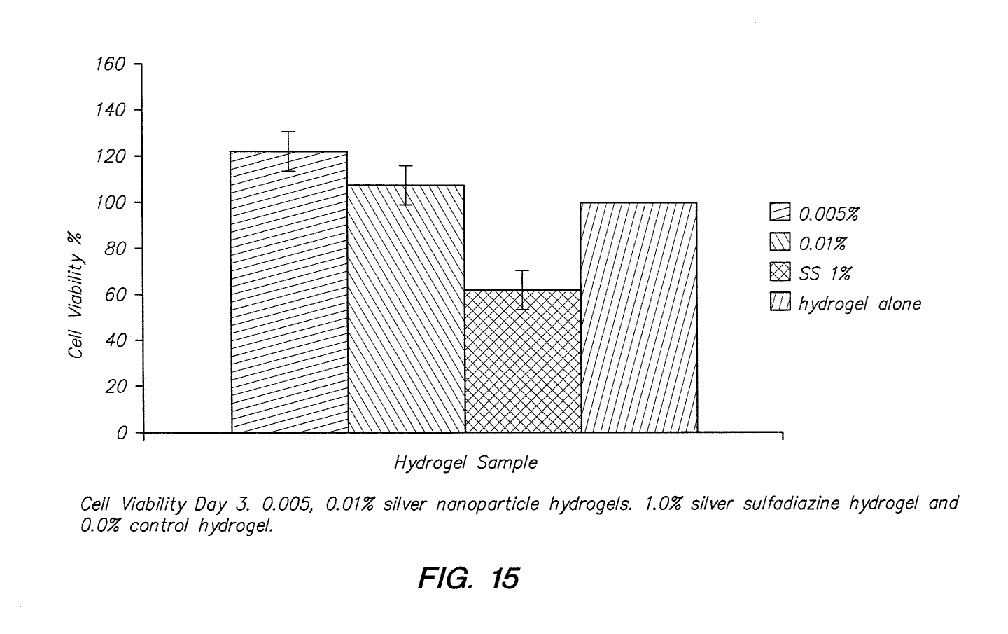

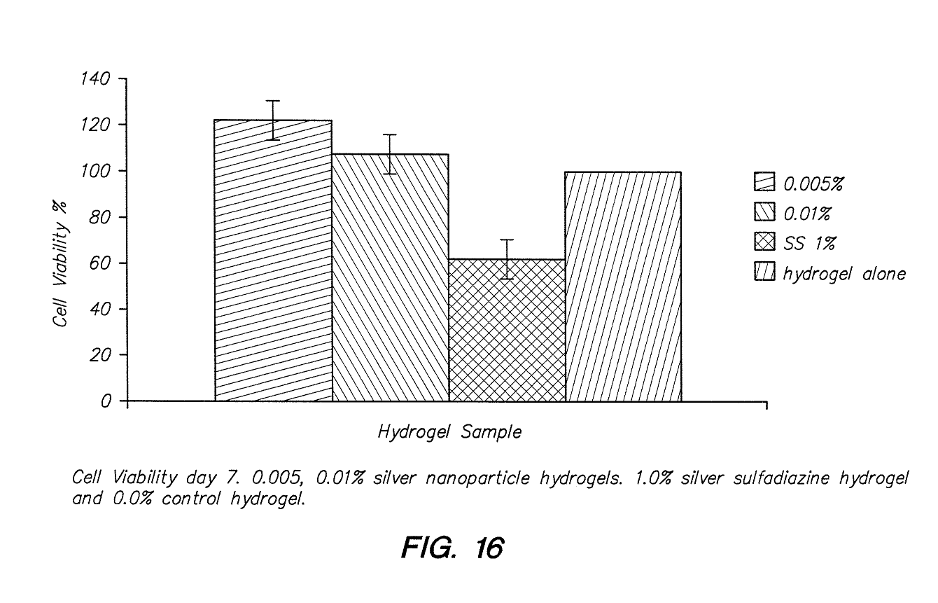

Cell viability was determined via Alamar Blue assay of hydrogel samples containing both Silver Nanoparticles and Silver Sulfadiazine powder. Cell viability was assessed on days 1, 4 and 7 for both Silver nanoparticles and Silver Sulfadiazine Powder. From FIG. 14, FIG. 15 and FIG. 16, it is clear that these hydrogel samples containing silver based agents are safe to use with cells. At day 1 it is clearly visible that the silver nanoparticle hydrogel samples have not affected the ADSC's. Following day 3, proliferation of cells occurs. This shows that these hydrogel samples containing silver nanoparticles neither damage nor inhibit the growth of these cells.

Silver sulfadiazine hydrogel at 1% was also used in the assay. This is a more powerful anti-bacterial agent and with that comes more potency towards cells. At day 1 a reduction in the number of cells was observed but because there is still a healthy percentage cell viability at 65%, this can be concluded safe for cell work. Further cell viability studies with reduced concentrations of SSD can be tested to increase this cell viability. Furthermore, it was observed that at day 3 the viability of these cells did not drop from 65%.

The cell viability assay conclusively demonstrated that these silver nano and silver sulfadiazine particles can be used and selectively target pathogenic cells with no effect on the live cells for the silver nanoparticles. This study also showed that use of silver sulfadiazine can affect live cells and dictates that further cell viability assays be performed with lower concentrations of SSD to ensure the wound care dressing does not delay regeneration.

Although the use of a hyperbranched PEG-based copolymer according to the present teaching has mainly been described with reference to its use for hydrogels and wound dressings, it will be appreciated that the copolymer described may also be used for a wide variety of other applications. For example, it may be used as a vehicle for drug delivery, for example for targeted therapy of diseases. For example, using conjugation techniques therapeutic components could be conjugated to the vinyl end groups of the copolymer system and injected into a patient for targeted therapy of diseases.

The words comprises/comprising when used in this specification are to specify the presence of stated features, integers, steps or components but does not preclude the presence or addition of one or more other features, integers, steps, components or groups thereof.

While the particular Multifunctional Hyperbranched Polymers as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A hyperbranched copolymer comprising PEG-based monomers wherein the hyperbranched copolymer comprises a double bond content in the range 5 mol % to 50 mol %.

2. A hyperbranched copolymer according to claim 1, wherein the copolymer comprises a double bond content in the range 10 mol % to 25 mol %, preferably at least 24 mol %.

3. A hyperbranched copolymer according to claim 1, wherein the copolymer comprises a degree of branching in the range 5 mol % to 30 mol %, preferably at least 27 mol %.

4. A hyperbranched copolymer according to claim 1, wherein use of the copolymer is selected from the group consisting of a drug delivery vehicle, a diagnostic tool, a cross-linking agent, a cross-linking agent in the preparation of a 3 dimensional printable material, and an additive.

5. A hyperbranched copolymer according to claim 1, wherein the PEG-based monomers have molecular weights in the range 1000 Da to 500 kDa.

6. A hyperbranched copolymer according to claim 1, wherein the PEG based monomers comprise end functional groups selected from the group consisting of acrylate, methacrylate, hydroxyl, carboxyl, amine, thiol, aldehyde, azide and sulphone and wherein the PEG-based monomers are selected from the group consisting of PEGDA ((poly)ethylene glycol)diacrylate), PEGAcryate ((poly)ethylene glycol)acrylate), PEGMEMA (poly(ethylene glycol)methyl ether methacrylate), PEGDMA (poly(ethylene glycol)dimethacrylate) and PEG methacrylate (poly(ethylene glycol)methacrylate).

7. A hyperbranched copolymer according to claim 6, comprising a PEGDA-PEGMEMA copolymer for selective use as a hydrogel for a wound dressing, a topical treatment, an antibacterial composition, and a treatment for mastitis.

8. A hyperbranched copolymer according to claim 6, wherein one or more agents are conjugated to said end functional groups and wherein the agent is selected from the group consisting of biomolecules, proteoglycans, fibres, glycoproteins, non-proteoglycan polysaccharides, antimicrobial agents, antibacterial agents and drugs.

9. A hyperbranched copolymer according to claim 1, wherein the hyperbranched polymers are crosslinkable in situ to form a hydrogel and the copolymer has a shelf life of at least 12 months.

10. A hyperbranched copolymer according to claim 1, wherein the hyperbranched polymers are synthesized by copolymerising the PEG-based monomers using RAFT (Reverse Addition-Fragmentation chain Transfer) polymerisation.

11. A hyperbranched copolymer according to claim 10, wherein the RAFT agent is selected from the group consisting of Dithiobenzoates, Trithiocarbonates and Dithiocarbamates.

12. A hyperbranched copolymer according to claim 10, wherein the PEG-based monomers comprise PEGDA and PEGMEMA and wherein the initial monomer feed ratio of PEGDA and the PEGMEMA is in the range 25 mol % to 75 mol %, preferably 50 mol % of the total feed ratio of monomers.

13. A hyperbranched copolymer according to claim 12, wherein the hyperbranched copolymer comprises a PEGDA-PEGMEMA copolymer and further wherein the PEGDA-PEGMEMA copolymer is combined with a cross-linking agent to form a hydrogel.

14. A hyperbranched copolymer according to claim 13, wherein the crosslinking agent is selected from the group consisting of thiolated hyaluaronan, pentaerythritol tetrakis 3-mercaptopropionate (QT), chitosan, gelatine, thiolated chitosan, fibrinogen or any natural thiolated biomolecule.

15. A two part hydrogel composition comprising (i) a crosslinking agent and (ii) a polymeric solution comprising a hyperbranched PEG-based copolymer comprising PEG-based monomers wherein the hyperbranched copolymer comprises a double bond content in the range 5 mol % to 50 mol %.

16. A two part hydrogel composition according to claim 15, wherein the polymeric solution comprises an in situ cross-linkable PEGDA-PEGMEMA copolymer.

17. A two part hydrogel composition according to claim 15 wherein the crosslinking agent is selected from the group consisting of thiolated hyaluaronan, pentaerythritol tetrakis 3-mercaptopropionate (QT), thiolated chitosan, fibrinogen, gelatine, chitosan or any natural thiolated biomolecule and the polymeric solution further comprises an agent selected from the group consisting of biomolecules, antimicrobial agents, antibacterial agent, anti-fungal agent, anti-viral agent, drug, agent for the regeneration of tissue, fibrinogen or a gene.

18. A two part hydrogel composition according to claim 17 wherein the antibacterial agent is selected from the group consisting of silver nanoparticles, colloidal silver, silver sulfadiazine, antibiotics, honey, copper, zinc, hydrogen peroxide and antimicrobial peptides (AMP's).

19. (canceled)
